(19) United States
(12) Patent Application Publication
(10) Pub. No.: US 2009/0117160 A1
(43) Pub. Date: May 7, 2009

(54) Compositions for Making up Keratinous Materials

(75) Inventors: Christophe Dumousseaux, Tokyo (JP); Makoto Kawamoto, Kanagawa (JP)

Correspondence Address: OLIFF & BERRIDGE, PLC, P.O. BOX 320850, ALEXANDRIA, VA, 22320-4850, US

(73) Assignee: L 'OREAL, Paris (FR)

(21) Appl. No.: 11/922,004

(22) PCT Filed: Jun. 22, 2006
(86) PCT No.: PCT/FR2006/001437

§ 371 Date: Mar. 13, 2008

Related U.S. Application Data
(60) Provisional application No. 60/692,585, filed on Jun. 22, 2005.
(60) Provisional application No. 60/758,545, filed on Jan. 13, 2006.

(51) Int. Cl. A61K 8/02; A61K 31/74; A61Q 1/00; A61K 8/30

(52) U.S. Cl. 424/401; 424/78.03; 424/63

(57) ABSTRACT

The present invention relates to a kit comprising:
a first cosmetic composition comprising:
   a physiologically-acceptable medium; and
   monodisperse particles suitable for forming an ordered lattice of monodisperse particles on a substrate on which the composition is applied; and
a second cosmetic composition including a polymer, said second composition being for application under a layer of first composition.

The present invention relates to cosmetic compositions, and more particularly to those for making up keratinous materials, in particular the skin, the lips, the nails, the eyelashes, and the hair.

### BACKGROUND

It is known to use pigments and colorants in makeup compositions.

The use of such pigments and colorants can nevertheless give rise to difficulties.

Thus, pigments and colorants can present relatively poor resistance to ultraviolet radiation and can spoil in light.

In addition, when color is provided by an absorption phenomenon, the coloring produced can be less vivid and bright than desired.

Finally, the choice of pigments and colorants that are suitable for use in cosmetics can be found to be insufficient.

Pigments and colorants can also impose constraints on formulation.

In order to obtain a goniochromatic effect, it is known to use interference pigments. These are nevertheless relatively complex and expensive to fabricate.

A goniochromatic effect present in a formulation can also be provided by an ordered lattice of monodisperse particles, as taught in particular in application WO 00/47167.

In spite of the relative age of that publication, so far as the Applicant is aware, there still does not exist on the market at present any cosmetic that enables vivid and bright coloring to be obtained for a duration that is acceptable to the consumer by using an ordered lattice of monodisperse particles after they have been applied on keratinous materials.

Publication WO 02/056854 in the name of the Applicant company discloses an iridescent composition for topical application comprising at least one hydrosoluble wetting agent and monodisperse particles in aqueous dispersion, those particles having a number mean size lying in the range 50 nanometers (nm) to 300 nm, with the quantity of those particles constituting at least 3% by weight relative to the total weight of the composition.

Application WO 05/018566 discloses a topical system for application to the skin, comprising a colloidal crystal lattice in a hydrophilic phase and at least one phase containing an oil.

### SUMMARY

There exists a need to further improve compositions enabling a color to be produced using at least one ordered lattice of monodisperse particles, with such a lattice sometimes being referred to as a “photonic crystal”.

In particular, there exists a need to improve the visibility of the color produced in this way and/or to make it easier to form the lattice and/or to improve its retention on keratinous materials.

The invention seeks to satisfy at least one those needs. Some embodiments of the invention that are described in detail below can satisfy all of those needs, and others can satisfy only some of them.

In one of its aspects, the invention thus provides a kit comprising:
a first composition comprising:
   a physiologically-acceptable medium; and
   monodisperse particles suitable for forming an ordered lattice of monodisperse particles on a substrate on which the first composition is applied; and
a second composition preferably including a polymer, said second composition being for application under a layer of first composition.

The percentage by weight of monodisperse particles in the first composition may be greater than 15%, for example, better greater than 20%. Such a concentration can make it easier to form a crystal lattice, e.g. with the help of a cosmetic applicator. A relatively high concentration can lead to particles being pre-organized by electrostatic repulsion within the composition, or after it has dried. Other percentages by weight are possible without going beyond the ambit of the present invention.

The particles may optionally form a compact crystal lattice after application. The lattice may possibly be discontinuous, presenting fractures and dislocations.

An approximation for the wavelength λ of the light that is diffracted by the lattice is given by Bragg's law:

mλ=2nd sin θ

where m is the diffraction order, n is the mean refractive index of the diffracting medium, d the distance between two diffracting planes, and θ the Bragg angle between the incident light and the diffracting plane.

The diffracted wavelength then depends mainly on the angle of observation and the distance between the particles. When the lattice that is formed is compact, this distance depends mainly on the size of the particles. It is thus possible to obtain different goniochromatic colorings by varying the size of the particles present.

It is also possible to obtain reflection in the ultraviolet (UV) range (for protection against UVs), or in the infrared (IR) range (antiheat coating).

Since the distance between the particles varies during drying, it is relatively easy to obtain cosmetic compositions that present continuous variation in color (from red to blue) during drying after application, which can lead to a playful effect for the consumer.

If so desired, the invention makes it possible to provide a cosmetic composition that does not have any colorant or pigment, with color being produced by the ordered lattice of monodisperse particles.

The invention also makes it possible to produce a colored deposit that is sensitive to an external stimulus, such as, for example: temperature; humidity; or ultraviolet radiation.

Such a stimulus can exert an influence on the distance between the particles of the lattice, and thus modify the color, as explained above.

The distance between the particles can be modified, e.g. by varying the size of the particles under the effect of the external stimulus, and/or by varying the distance between particles of substantially constant size, e.g. by varying the forces of repulsion between them, and/or by varying the size of at least one compound that is present between the particles.

The refractive index of the medium may optionally vary under the effect of the external stimulus, e.g. temperature.

The invention also makes it possible to produce, where appropriate, a deposit having a color that changes as a function of the degree to which the composition has dried.

The invention makes it possible to obtain coloring that is durable and bright over a large area.

### Monodisperse Particles

The term “monodisperse particles” is used in the invention to designate particles of mean size presenting a coefficient of variation CV that is less than or equal to 15%.

The coefficient of variation CV is defined by the relationship:

$$C V = \frac{s}{D}$$

where s is the standard deviation of the size distribution of the particles, and D is the mean size thereof.

The mean size D and the standard deviation s can be measured on 250 particles by analyzing an image obtained with the help of a scanning electron microscope, e.g. the microscope referenced S-4500 from the supplier Hitachi. Image analysis software can be used for facilitating the measurement, e.g. Winroof® software sold by the supplier Mitani Corporation.

The coefficient of variation of the monodisperse particles is preferably less than or equal to 10%, better less than or equal to 7%, better still less than or equal to 5%, for example being substantially about 3.5%. Small dispersion in particle size can be favorable to the quality of the compact crystal lattice that is formed, and thus to obtaining colors that are vivid and glossy.

The mean size D of the monodisperse particles may generally lie in the range 80 nm to 500 nm, better in the range 100 nm to 500 nm, or 150 nm to 450 nm, possibly being selected as a function of the color(s) to be obtained and of the surrounding medium, for example.

A preferred mean size range is 150 nm to 450 nm, better 190 nm to 310 nm for obtaining colors in the visible range. The mean size may lie in the range 80 nm to 200 nm for UV filtering.

According to an aspect of the invention, the monodisperse particle content by weight may, for example, lie in the range 15% to 70%, e.g. being greater than 20%, 25%, 30%, 35%, 40%, or 45%. A different content, e.g. lying in the range 1% to 70% can be acceptable in certain other aspects of the invention.

Depending on the concentration of particles used in the composition, the periodic lattice that is formed may be a single layer or a multilayer lattice, and it may be compact or otherwise.

The shape of the monodisperse particles must be compatible with forming an ordered lattice of monodisperse particles.

The ordered lattice may be at least partially body-centered cubic, face-centered cubic, compact hexagonal, or hybrid, being made up of those arrangements or others.

Various examples of crystal lattice formation from monodisperse particles are given in the publication by Xia et al., Adv. Mater., 12, 693-713 (2000).

Preferably, the monodisperse particles are spherical in shape, however other shapes are possible, in particular those presenting axial symmetry.

The monodisperse particles may be simple materials or composites.

The monodisperse particles may be solid or hollow. Hollow monodisperse particles present density that is less than that of solid particles and thus make it possible to occupy more volume for a given concentration by weight. When the monodisperse particles are constituted by a high density material, e.g. an inorganic material, hollow particles make it possible to limit phenomena of settling in the composition.

The presence of air or some other gas inside the particles after drying makes it possible to obtain a large difference in refractive index between the particles and the surrounding medium, which is favorable in terms of the density of the diffraction peak and thus in terms of developing coloring that is very intense. Numerous non-volatile compounds can be added into the composition or onto the composition without running any risk of losing color and of ending up with a composition that is transparent.

The monodisperse particles may optionally be porous. The presence of pores of small size within the particles can reduce the refractive index of the particles.

The refractive index nₚ of the monodisperse particles is different from the refractive index n_{c} of the continuous medium extending around the particles after the formulation has been applied, and the difference between these refractive indices is preferably greater than or equal to 0.02, better greater than or equal to 0.05, better still greater than or equal to 0.1, e.g. lying in the range 0.02 to 2, and in particular in the range 0.05 to 1.

Too small a refractive index difference nₚ-n_{c} can require a large number of layers of particles in the ordered lattice in order to obtain the desired result. Too great an index difference can accentuate phenomena of light diffusion within the layer and can lead to the deposit whitening after it has been applied.

The refractive index of monodisperse particles is defined as being the mean refractive index. With composite particles, it is calculated in linear manner as a function of the proportion by volume of each component.

The refractive index of the monodisperse particles can be greater than or equal to that of the medium, e.g. being greater than or equal to 1.4, in particular lying in the range 1.4 to 1.7.

All of the monodisperse particles corresponding to a given mean size D may have substantially the same refractive index.

The monodisperse particles may be colored, i.e. not white, e.g. in order to reinforce the intensity of the color produced and/or to avoid a phenomenon of the composition whitening after being applied onto keratinous materials.

An example of a colored particle used to form a colloidal crystal is given in publication WO 05/012961.

The color of the monodisperse particles can be provided by selecting the material(s) constituting each monodisperse particle. It may have the effect of increasing the absorption of light by the particles and of diminishing diffusion.

The monodisperse particles may in particular incorporate at least one pigment or colorant that is organic or inorganic, possibly being fluorescent and presenting ultraviolet or infrared fluorescence, where appropriate.

The monodisperse particles may include an inorganic compound, or may even be completely inorganic.

When the monodisperse particles are inorganic, they may for example include at least one oxide, in particular a metal oxide, e.g. being selected from the oxides of: silicon; iron; titanium; aluminum; chromium; zinc; copper; zirconium; and cerium; and mixtures thereof. The monodisperse particles may also comprise a metal, in particular: titanium; silver; gold; aluminum; zinc; iron; copper; and mixtures and alloys thereof.

The monodisperse particles may include an organic compound, or they may be entirely organic.

Amongst materials that can be suitable for making organic monodisperse particles, mention can be made of polymers, in particular carbon or silicone chain polymers, e.g. polystyrenes (PS), polymethylmethacrylate (PMMA), polyacrylamide (PAM), and silicone polymers.

The monodisperse particles may include at least one polymer or copolymer suitable for ionizing in order to improve dispersability in the medium and electrostatic stabilization. In aqueous solution, this polymer or copolymer preferably contains carboxylic or sulfonic acid functions.

When the monodisperse particles are composite, they may for example comprise a core and a “husk” made of different materials, e.g. organic and/or inorganic materials.

When the monodisperse particles are composite, the core material or the husk material may be selected for example in order to improve stability in the monodisperse particle medium, to increase refractive index, and/or to color the particles, and/or to impart fluorescence or magnetic susceptibility thereto.

The core may be constituted by a material that is insoluble in the medium containing the particles, e.g. an inorganic material, such as silica, for example, or an organic material, such as an acrylic polymer, for example.

The husk may be constituted by polymer chains, which may be soluble in the medium containing the particles, the polymer chains possibly including polymers grafted to the surface of each monodisperse particle core, which itself may be insoluble in the medium.

Such particles having a core and polymer chains, are also known as “hairy” particles, and they can be stabilized in the medium not only by electrostatic interactions, but also by steric interactions of the excluded volume type.

The additional stabilization and volume provided by the polymer chains make it easy to incorporate other components in the composition without any risk of destabilization and of particles clumping. These other components may, for example, be coloring agents or fillers intended to modify the appearance of the composition or of the substrate covered therewith, for example.

The polymer chains may include grafted polymer chains that may contain chemical functions (carboxylic acid, amine, amide, thiol, . . . ) suitable for integrating with keratinous materials and for improving the adhesion of the composition on the covered substrate.

The polymer chains may also improve the retention of the particle lattice after application on the keratinous materials.

By way of example, examples of “hairy” particles are given in the publication by Ishizu et al., Kagaku To Kogy, 57(7) (2004) for a polymer core, or in the publication by Okubo et al., Colloid & Polymer Science, 280(3), pp. 290-295 (2002) for a silica core with polymethylmethacrylate or poly(styrene co maleic anhydride) polymers in the husk.

Another example of “hairy” particles is given in the publication by Tsuji et al., Langmuir, 21, pp. 2434-2437 (2005) for a polystyrene core with poly N isopropyl acrylamide hairs.

Where appropriate, the presence of a husk can serve to encapsulate therein a compound for which direct contact with keratinous materials or the medium is not desirable.

The composite monodisperse particles may also comprise inclusions of a first material in a matrix of a second material. For example, the first material may present a high refractive index enabling the overall refractive index of the particles to be increased. By way of example, the particles may have inclusions of nanoparticles, e.g. nanoparticles of titanium oxide.

The monodisperse particles may be fabricated using methods of synthesis as described for example in the publication by Xia et al., Adv. Mater., 12, 693-713 (2000) incorporated herein by reference.

As commercial references for monodisperse particles that can be suitable, mention can be made of Seahoster® KE-W10 (silica), Seahoster® KE-W20 (silica), Seahoster® KE-W25 (silica), Seahoster® KE-W30 (silica), Seahoster® KE-P20 (silica), Seahoster® KE-P30 (silica) from the supplier Nippon Shokubai, 216 nm or 290 nm Optibind® (polystyrene) microparticles from the supplier Seradyn, Cosmo® 30 (silica) from the supplier CCIC, Hipresica® FQ (silica) from the supplier Ube-Nitto, Eposter® MX-100W (PMMA) and Eposter® MX-200W (PMMA) from the supplier Nippon Shokubai.

Examples of magnetic particles and of monodisperse particles based on silica are described in the article by Xu et al., Chem. Mater., 14, 1249-1256 (2002).

Where appropriate, the monodisperse particles may present a dimension that is sensitive to an external stimulus, e.g. concentration of a compound and/or temperature and/or pressure.

By way of example, the monodisperse particles are particles of a polymer that have swelled in a solvent, the particles forming a microgel.

The publication by HU et al., Angevandte Chemie 42, 4799-4802 (2003) discloses poly-N-isopropylacrylamide-based particles and a method of obtained colloidal crystals with such particles. Such particles swell to a greater or lesser extent as a function of temperature, thereby making it possible to obtain a color that is sensitive to temperature. Poly-N-isopropylacrylamide-based polymers may also be present in a husk of monodisperse particles, in particular of “hairy” particles.

### Medium Containing Monodisperse Particles

According to the invention, the monodisperse particles may be contained, at least prior to application, in a physiologically acceptable medium enabling an ordered lattice of monodisperse particles to be formed on the substrate on which the composition is applied.

The term “physiologically acceptable medium” is synonymous to the term “cosmetically acceptable medium” and is used to mean a non-toxic medium suitable for being applied on the keratinous materials of human beings, in particular the skin, the mucous membranes, the nails, or hair.

The physiologically acceptable medium is generally adapted to the nature of the substrate on which the composition is to be applied and also to the form in which the composition is to be packaged.

The monodisperse particles may be contained in a liquid phase.

The medium containing the monodisperse particles may be entirely liquid or it may contain other particles, where appropriate.

The medium may be selected in such a manner as to encourage the particles to disperse in the medium prior to application thereof, so as to avoid particles clumping.

The medium may be selected in such a manner that the ordered lattice of monodisperse particles is formed by the particles stacking in regular manner after the medium has been applied to keratinous materials, the lattice not existing in the composition prior to application and forming as a solvent contained in the composition evaporates, for example.

As mentioned above, the refractive index of the medium advantageously presents a difference relative to that of the monodisperse particles, the absolute value of said difference preferably being greater than or equal to 0.02, better greater than or equal to 0.05, in particular lying in the range 0.05 to 1, better still greater than or equal to 0.1.

The medium may be aqueous, the monodisperse particles being suitable for being contained in an aqueous phase. The term “aqueous medium” is used to mean a liquid medium at ambient temperature and atmospheric pressure that contains a large fraction of water relative to the total weight of the medium. The remaining fraction may contain or be constituted by physiologically-acceptable organic solvents that are miscible in water, e.g. alcohols or alkylene glycols. The percentage by weight of water in the aqueous medium is preferably greater than or equal to 30%, better 40%, still better 50%.

The medium may be single-phase or multiphase and may optionally include solids other than the monodisperse particles, in particular particles that are smaller or particles that are larger.

Preferably, in the presence of solid bodies other than the monodisperse particles, the quantity of such bodies is sufficiently small to avoid impeding the formation of the ordered lattice of monodisperse particles and to avoid impeding obtaining the result that is desired, in particular in terms of coloring.

The medium may include at least one compound presenting an OH bond, in particular an alcohol function, at a percentage by weight that is greater than or equal to 5%, or better greater than or equal to 10%, for example. Such a compound can slow down evaporation without disturbing the formation of an ordered lattice.

The medium may include an alcohol such as ethanol, or isopropanol, for example, or a glycol derivative, in particular ethylene glycol or propylene glycol.

The medium preferably presents a relative dielectric constant ∈ that is greater than or equal to 10, better greater than or equal to 20, still better greater than or equal to 30. The dielectric constant is measured at a temperature of 25° C. A relatively high dielectric constant encourages the monodisperse particles to become ordered in a lattice.

The conductivity of the composition may lie in the range 5 micro siemens per centimeter (μS.cm⁻¹) to 2000 μS.cm⁻¹, in particular in the range 10 μS.cm⁻¹ to 4000 μS.cm⁻¹, or even in the range 20 μS.cm⁻¹ to 400 μS.cm⁻¹.

The medium may be transparent or translucent, colored or otherwise. The medium containing the monodisperse particles need not contain any pigment or colorant. The coloring of the medium may correspond to adding an additional coloring agent.

By way of example, the color of the medium may correspond to one of the colors that can be generated by the ordered lattice of monodisperse particles, e.g. the color produced by the lattice when observed under normal incidence.

The color of the medium may also be black so as to limit the diffusion of light.

The ordered lattice of monodisperse particles can make it fairly easy to obtain green, red, or blue colors. The color range can be extended by the presence of an additional coloring agent, e.g. a colorant, an absorbent pigment, or an effect pigment, e.g. at a concentration lying in the range 0.1% to 15% by weight.

The term “effect pigment” is used to cover, amongst others: reflecting particles; nacres; goniochromatic coloring agents; or diffracting pigments; as defined below.

The presence of pigments of relatively large size, such as nacres for example, need not prevent the lattice forming beside the pigment particles, and on the contrary it can encourage such formation by improving the confinement of the monodisperse particles, where the larger particles can become inserted in certain dislocations of the lattice.

The medium can thus include larger particles having a size that is at least three and better five times greater than the size of the monodisperse particles, and better still ten times greater.

These larger particles may be particles of a non-coloring filler or pigment.

The medium may thus include at least one effect pigment.

The presence of monodisperse particles makes it possible to obtain a periodic lattice after application onto keratinous materials. The lattice enables a coloring effect to be obtained by diffracting light, and the Applicant has found that it is possible to associate a second optical effect by means of an effect pigment while conserving the periodic lattice. These two optical effects are additive, and the presence of the pigment thus makes it possible to extend the color range and the optical effects obtained by the lattice formed on the keratinous materials.

The effect pigment may be present in the formulation at a concentration lying in the range 0.1% to 70%, preferably in the range 1% to 50%, more preferably in the range 5% to 20%.

### Reflective Particles

Reflective particles can serve to create highlights that are visible to the naked eye.

The reflective particles may be present in a variety of forms. The particles may in particular be in the form of platelets or they may be globular, in particular spherical. The particles may comprise a substrate covered in a reflective material.

The substrate may be selected from: glasses; metallic oxides; aluminas; silicas; silicates, in particular aluminosilicates and borosilicates; mica; synthetic mica; synthetic polymers; and mixtures thereof.

The reflective material may include a layer of metal or of a metal compound.

Particles having a substrate of glass coated in silver in the form of platelets are sold under the name Metashine by the supplier Nippon Sheet Glass.

By way of example of reflective particles, mention can also be made for example of: particles comprising a synthetic mica substrate coated in titanium dioxide; or particles of glass coated either in: brown iron oxide; titanium oxide; tin oxide; or a mixture thereof, such as those sold under the trademark Reflecks® by the supplier Engelhard.

Also suitable for the invention are pigments from the Metashine 1080R range sold by the supplier Nippon Sheet Glass Co. Ltd. These pigments are more particularly described in patent application JP 2001-11340, and they are constituted by flakes of C-GLASS glass comprising 65% to 72% of SiO₂, covered in a layer of titanium oxide of the rutile type (TiO₂). These glass flakes have a mean thickness of 1 micrometer (μm) and a mean size of 80 μm, giving a ratio of mean size divided by mean thickness of 80. They present a blue, green, yellow, or silvery sheen depending on the thickness of the TiO₂ layer.

Mention can also be made of particles of size lying in the range 80 μm to 100 μm, comprising a substrate of synthetic mica (fluorophylogopite) coated in titanium dioxide representing 12% of the total weight of the particle, sold under the name Prominence by the supplier Nihon Koken.

The reflective particles may also be selected from particles formed by stacking at least two layers having different refractive indices. Such layers may be of polymeric or metallic nature and in particular they may include at least one polymeric layer. Thus, the reflective particles may be particles derived from a multilayer polymeric film. Such particles are described in particular in WO 99/36477, U.S. Pat. No. 6,299,979, and U.S. Pat. No. 6,387,498. Reflective particles comprising a stack of at least two polymer layers are sold by the supplier 3M under the name Mirror Glitter. Those particles have layers of 2,6-PEN [polyethylene naphthalate] and of polymethyl methacrylate in a weight ratio of 80/20. Such particles are described in U.S. Pat. No. 5,825,643.

### Nacres

The term “nacres” is used to mean colored particles of any shape, presenting an optical interference color effect and optionally iridescent, in particular those produced in the shells of certain mollusks, or else those that are synthesized.

Nacres can be selected from nacre pigments such as: titanium mica covered in an iron oxide; mica covered in bismuth oxychloride; titanium mica covered in chromium oxide; titanium mica covered in an organic colorant, in particular a colorant of the above-specified type; and nacre pigments based on bismuth oxychloride. They could also be particles of mica having at least two successive layers of metallic oxides and/or organic coloring materials superposed on their surfaces.

As examples of nacres, mention can also be made of natural mica covered in: titanium oxide; iron oxide; natural pigment; or bismuth oxychloride.

Amongst the nacres available on the market, mention can be made of the Flamenco nacres sold by the supplier Engelhard, and the Timiron nacres sold by the supplier Merck.

### Goniochromatic Coloring Agents

Coloring agents that are goniochromatic in the meaning of the present invention present a color change, also known as a “color flop”, as a function of the angle of observation that is greater than that encountered with nacres.

By way of example, the goniochromatic coloring agent may be selected from interference multilayer structures and liquid crystal coloring agents.

Examples of symmetrical interference multilayer pigments suitable for use in compositions made in accordance with the invention are, for example: Chromaflair from the supplier Flex; Sicopearl from the supplier Basf; Xirona pigments from the supplier Merck (Darmstadt); Infinite Colors pigments from the supplier Shiseido; and Color Relief pigments from the supplier CCIC.

It is also possible to use goniochromatic coloring agents of multilayer structure comprising alternating polymeric layers, e.g. of the polyethylene naphthalate and polyethylene terephthalate type. Such agents are described in particular in WO-A-96/19347 and WO-A-99/36478.

As examples of pigments having a polymeric multilayer structure, mention can be made of those sold by the supplier 3M under the name Color Glitter or those sold by the supplier Venture Chemical under the name Micro Glitter Pearl.

By way of example, liquid crystal coloring agents comprise silicones or cellulose ethers on which mesomorphic groups have been grafted. As liquid crystal goniochromatic particles, use can be made for example of those sold by the supplier Chemx and also those sold under the name Helicone® HC by the supplier Sicpa.

The composition may also include dispersed goniochromatic fibers. Such fibers may for example present a size lying in the range 50 μm to 2 mm.

Goniochromatic fibers having a two-layer structure of polyethylene terephthalate and nylon-6 are sold by the supplier Teijin under the names Morphotex and Morphotone.

### Diffracting Pigments

The term “diffracting pigments” is used to mean a pigment having a periodic motif constituting a diffraction grating. Since the distance between the periodic motifs is of the same order of magnitude as the wavelength of visible light, the pigments can diffract light and produce a rainbow effect, for example.

Such pigments are commercially available under the name Spectraflair from the supplier JDS Uniphase Corporation.

Such pigments can also be made using the methods taught by the following patents: U.S. Pat. No. 6,818,051; U.S. Pat. No. 6,894,086; and EP 1 634 619. Those patents describe pigments constituted by a three-dimensional lattice of silica particles similar in structure to opals. Inverse opal structures can also be obtained and used.

The medium in which the ordered lattice of monodisperse particles forms may optionally evaporate after the composition has been applied.

Preferably, the medium includes a volatile solvent. The term “volatile solvent” is used in the meaning of the invention to designate any liquid suitable for evaporating on contact with the skin at ambient temperature and at atmospheric pressure.

The medium may be selected in particular in such a manner that the composition contains at least 10%, or even at least 30% volatile solvent.

The pH of the composition may lie in the range 1 to 11, e.g. in the range 3 to 9. The pH most adapted to the formation of the lattice may depend on the nature of the monodisperse particles. A basic pH is preferred when the monodisperse particles are inorganic, in particular including silica.

The medium may include smaller particles having a mean size D that is less than that of the monodisperse particles, being smaller by a factor of at least 2, better of at least 3, so as to enable them to become inserted in the voids left between the monodisperse particles of the lattice.

These interstitial particles may be inorganic or organic and can serve to improve the cohesion of the lattice or to modify the way light is absorbed by the layers of the lattice.

As examples of interstitial particles, mention can be made of nanoparticles of: titanium dioxide; silica; iron oxide; or of carbon black; presenting a mean size lying in the range 5 nm to 150 nm, e.g. lying in the range 10 nm to 100 nm.

As another example of interstitial particles, mention can be made of particles of a polymer, e.g. already in the polymerized state within the composition prior to application on keratinous materials, the medium including a latex, for example.

Where appropriate, the size of the interstitial particles may vary as a function of an external stimulus and/or as a function of the concentration of a compound in the medium. The interstitial particles may be hydroabsorbent. The size of the particles may for example then vary as a function of the concentration of water in the medium.

Where appropriate, the variation in the size of the interstitial particles may exert an action on the distance between the monodisperse particles, and thus have an action on the color produced by the lattice.

The medium may include at least one polymer for improving retention of the lattice after it has formed.

By way of example, before the composition is applied and has dried, the polymer may be in a state in which it is not fully polymerized and/or cross-linked.

When the medium contains a polymer that is not fully polymerized and/or cross-linked prior to application of the composition on keratinous materials, the cross-linking and/or polymerization can take place after the composition has been applied on the keratinous materials.

By way of example, the polymerization and/or cross-linking can occur after the lattice of monodisperse particles has formed, or in a variant beforehand, and/or at the same time.

The medium may include a film-forming polymer.

### Film-Forming Polymer

In the present invention, the term “film-forming polymer” is used to mean a polymer suitable, on its own or in the presence of an auxiliary film-forming agent, for forming a macroscopically continuous film that adheres on keratinous materials, and preferably a film that is cohesive, and better still a film presenting cohesion and mechanical properties that are such that such film can be isolated and handled in isolation, e.g. when said film is formed by casting onto a non-stick surface such as a Teflon or silicone surface.

The composition may include an aqueous phase and the film-forming polymer may be present in the aqueous phase. In this event, said film-forming polymer is preferably a polymer in dispersion or a polymer that is amphiphilic or associative.

The term “polymer in dispersion” is used to mean polymers that are not soluble in water and that are present in the form of particles of various sizes. The polymer may optionally be cross-linked. The mean particle size lies typically in the range 25 nm to 500 nm, preferably in the range 50 nm to 200 nm. The following polymers in aqueous dispersion can be used: Ultrasol 2075 from Ganz Chemical; Daitosol 5000AD from Daito Kasei; Avalure UR 450 from Noveon; Dynamx from National Starch; Syntran 5760 from Interpolymer; Acusol OP 301 from Rohm & Haas; and Neocryl A 1090 from Avecia.

Acrylic dispersions sold under the trade names: Neocryl XK-90®, Neocryl A-1070®, Neocryl A-1090®, Neocryl BT-62®, Neocryl A-1079®, and Neocryl A-523® by AVECIA-NEORESINS; Dow Latex 432® by DOW CHEMICAL; Daitosol 5000 AD®, or Daitosol 5000 SJ® by DAITO KASEY KOGYO; Syntran 5760® by Interpolymer; Allianz OPT by ROHM & HAAS; aqueous dispersions of acrylic or styrene/acrylic polymers sold under the trade name JONCRYL® by JOHNSON POLYMER; or else aqueous polyurethane dispersions sold under the trade name Neorez R-981®, and Neorez R-974® by AVECIA-NEORESINS; Avalure UR-405®, Avalure UR-410®, Avalure UR-425®, Avalure UR-450®, Sancure 875®, Sancure 861®, Sancure 878®, and Sancure 2060® by GOODRICH; Impranil 85® by BAYER; Aquamere H-1511® by HYDROMER; sulfopolyesters sold under the trade name Eastman AQ® by Eastman Chemical Products; vinyl dispersions such as Mexomere PAM® by CHIMEX; and mixtures thereof; are other example of aqueous dispersion of hydrodispersible film-forming polymer particles.

The term “polymers that are amphiphilic or associative” is used to mean polymers including one or more hydrophilic portions that make them partially soluble in water and one or more hydrophobic portions enabling the polymers to associate or interact. The following associative polymers can be used: Nuvis FX1100 by Elementis; Aculyn 22, Aculyn 44, Aculyn 46 by Rohm & Haas; or Viscophobe DB1000 by Amerchol. Diblock copolymers constituted by a hydrophilic block (polyacrylate, polyethylene glycol), and by a hydrophobic block (polystyrene, polysiloxan) can also be used.

Polymers that are soluble in an aqueous phase containing the monodisperse particles should be avoided since they can cause the monodisperse particles to clump together. The film-forming polymer can thus be non-soluble in such a phase.

The composition may include an oily phase and the film-forming polymer may be present in the oily phase. The polymer may be in dispersion or in solution. Polymers of the non-aqueous dispersion (NAD) type or of the microgel type (e.g. KSGs) can be used, as can polymers of the polystyrene-polyamide (PS-PA) type or copolymers based on (Kraton, Regalite styrene).

As examples of non-aqueous dispersions of film-forming polymers that are lipodispersible in the form of a non-aqueous dispersion of polymer particles in one or more silicone and/or hydrocarbon oils and that can be stabilized on the surface by at least one stabilizing agent, in particular a sequenced, grafted, or statistical polymer, mention can be made of: dispersions of acrylics in isododecane such as Mexomere PAP® from the supplier Chimex; dispersions of a preferably acrylic grafted ethylene polymer in a liquid fatty phase, the ethylene polymer advantageously being dispersed in the absence of any additional stabilizer on the surface of the particles, as is described in particular in document Wo 2004/055081.

Amongst the film-forming polymers suitable for use in the composition of the present invention, mention can be made of synthetic polymers of the radical type or of the polycondensate type, polymers of natural origin, and mixtures thereof.

The term “radical film-forming polymer” is used to mean a polymer obtained by polymerizing unsaturated monomers, in particular ethylene-unsaturated monomers, each monomer being capable of homopolymerizing (unlike polycondensates).

The radical type film-forming polymers may in particular be vinyl polymers or copolymers, in particular acrylic polymers.

Vinyl film-forming polymers may be the result of polymerizing ethylene-unsaturated polymers having at least one acid group, and/or esters of said acid monomers, and/or amides of said acid monomers.

As a monomer carrying an acid group, it is possible to use α,β-ethylene-unsaturated carboxylic acids such as: acrylic acid; methacrylic acid; crotonic acid; maleic acid; and itaconic acid. It is preferable to use (meth)acrylic acid and crotonic acid, and more preferably (meth)acrylic acid.

Esters of acid monomers are advantageously selected from: esters of (meth)acrylic acid (also known as (meth)acrylates), in particular alkyl(meth)acrylates, in particular C₁-C₃₀ and preferably C₁-C₂₀ alkyl (meth)acrylates; aryl(meth)acrylates, in particular C₆-C₁₀ aryl(meth)acrylates; hydroxyaklyl(meth)acrylates, in particular C₂-C₆ hydroxyaklyl(meth)acrylates.

Amongst alkyl(meth)acrylates, mention can be made of: methyl methacrylate; ethyl methacrylate; butyl methacrylate; isobutyl methacrylate; ethyl-2 hexyl methacrylate; lauryl methacrylate; and cyclohexyl methacrylate.

Amongst hydroxyalkyl(meth)acrylates, mention can be made of hydroxethyl acrylate; 2-hydropropyl acrylate; hydroxethyl methacrylate; 2-hydroxypropyl methacrylate.

Amongst aryl(meth)acrylates, mention can be made of benzyl acrylate and of phenyl acrylate.

The particularly preferred (meth)acrylate acid esters are alkyl(meth)acrylates.

In the present invention, the alkyl group of the esters may either be fluorinated, or perfluorinated, i.e. some or all of the hydrogen atoms of the alkyl group are substituted by fluorine atoms.

As amides of acid monomers, mention can be made for example of: (meth)acrylamides, and in particular N-alkyl (meth)acrylamides, in particular C₂-C₁₂ alkyl (meth)acrylamides. Amongst N-alkyl(meth)acrylamides, mention can be made of: N-ethyl acrylamide; N-t-butyl acrylamide; N-T-octyl acrylamide; and N-undecylacrylamide.

Vinyl film-forming polymers can also result from homopolymerization or copolymerization of monomers selected from vinyl esters and styrene monomers. In particular, these monomers may be polymerized with acid monomers and/or esters thereof and/or amides thereof, such as those mentioned above.

As examples of vinyl esters, mention can be made of: vinyl acetate; vinyl neodecanoate; vinyl pivalate; vinyl benzoate; and vinyl t-butyl benzoate.

As styrene monomers, mention can be made of styrene and of alpha-methyl styrene.

Amongst film-forming polycondensates, mention can be made of: polyurethanes; polyesters; amide polyesters; polyamides; and epoxy ester resins, and polyureas.

Polyurethanes can be selected from: anionic, cationic, non-ionic, or amphoteric polyurethanes; acrylic polyurethanes; polyvinyl pyrolidone polyurethanes; polyester polyurethanes; polyether polyurethanes; polyureas; polyurea polyurethanes; and mixtures thereof.

In known manner, polyesters can be obtained by polycondensation of dicarboxylic acids with polyols, in particular diols.

The dicarboxylic acid may be aliphatic, aclicyclic, or aromatic. As examples of such acids, mention can be made of: oxalic acid; malonic acid; dimethylmalonic acid; succinic acid; glutaric acid; adipic acid; pimelic acid; 2,2-dimethylglutaric acid; azelaic acid; suberic acid; sebacic acid; fumaric acid; maleic acid; itaconic acid; phthalic acid; dodecanedioic acid; 1,3-cyclohexane-dicarboxylic acid; 1,4 cyclohexanedicarboxylic acid; isophthalic acid; terephthalic acid; 2,5-norbornane dicarboxylic acid; diglycolic acid; thiodipropionic acid, 2,5-naphthalene dicarboxylic acid; and 2,6-naphthalene dicarboxylic acid. These dicarboxylic acid monomers can be used alone or in combination of at least two dicarboxylic acid monomers. Amongst these monomers, it is preferable to select: phthalic acid; isophthalic acid; or terephthalic acid.

The diol may be selected from aliphatic, alicyclic, or aromatic diols. It is preferable to use a diol selected from: ethylene glycol; diethylene glycol; triethylene glycol; 1,3-propanediol; cyclohexane dimethanol; and 4-butanediol. As other polyols, it is possible to use; glycerol; pentaerythritol; sorbitol; and trimethylol propane.

The amide polyesters may be obtained in analogous manner to the polyesters by polycondensation of diacids with diamines or with amino alcohols. As diamines, it is possible to use: ethylene diamine; hexamethylene diamine; meta- or para-phenylene diamine. As an amino alcohol, it is possible to use monoethanol amine.

The polyester may further include at least one monomer carrying at least one —SO₃M group, with M representing a hydrogen atom, an NH⁴⁺ ammonium ion, or a metallic ion, such as an Na⁺, Li⁺, K⁺, Mg²⁺, Ca²⁺, Cu²⁺, Fe²⁺, or Fe³⁺ ion. In particular, it is possible to use a bifunctional aromatic monomer including such a —SO₃M group.

The aromatic core of the bifunctional aromatic monomer that also carries a —SO₃M group as described above may be selected from benzene, naphthalene, anthracene, diphenyl, oxydiphenyl, sulfonyldiphenyl, and methylene diphenyl cores, for example. Examples of bifunctional aromatic monomers that may be mentioned, and that also carry a —SO₃M group, include, sulfoisophthalic acid, sulfoterephthalic acid, sulfophthalic acid, 4-sulfonaphthalene-2,7-dicarboxylic acid.

In an example composition of the invention, the film-forming polymer may be a polymer dissolved in a liquid fatty phase comprising organic solvents or oils (the film-forming polymer is then said to be a liposoluble polymer). The liquid fatty phase preferably comprises a volatile oil, optionally mixed with a non-volatile oil.

By way of example of a liposoluble polymer, mention can be made of copolymers of vinyl ester (the vinyl group being directly connected to the oxygen atom of the ester group and the vinyl ester having a saturated, linear, or branched hydrocarbon radical with one to 19 carbon atoms bonded to the carbonyl of the ester group) and at least one other monomer which may be: a vinyl ester (different from the already-present vinyl ester); an α-olefin (having eight to 28 carbon atoms); an alkyl vinyl ether (in which the alkyl group has two to 18 carbon atoms); or an allyl or methallyl ester (having a saturated, linear, or branched hydrocarbon radical with one to 19 carbon atoms bonded to the carbonyl of the ester group).

These copolymers may be cross-linked with the help of agents that may be either of the vinyl type or else of the allyl or methallyl type, such as: tetraallyloxyethane; divinyl benzene; divinyl octane dioate; divinyl dodecane dioate; and divinyl octadecane dioate.

As examples of these copolymers, mention can be made of the following copolymers: vinyl acetate and allyl stearate; vinyl acetate and vinyl laurate; vinyl acetate and vinyl stearate; vinyl acetate and octadecene; vinyl acetate and octadecyl vinyl ether; vinyl propionate and allyl laurate; vinyl propionate and vinyl laurate; vinyl stearate and 1-octadecene; vinyl acetate and 1-dodecene; vinyl stearate and ethyl vinyl ether; vinyl propionate and cetyl vinyl ether; vinyl stearate and allyl acetate; vinyl dimethyl-2,2 octanoate and vinyl laurate; allyl dimethyl-2,2 pentanoate and vinyl laurate; vinyl dimethyl propionate and vinyl stearate; allyl dimethyl propionate and vinyl stearate; vinyl propionate and vinyl stearate, cross-linked with 0.2% divinyl benzene; vinyl dimethyl propionate and vinyl laurate cross-linked with 0.2% divinyl benzene; vinyl acetate and octadecyl vinyl ether, cross-linked with 0.2% tetraallyl oxyethane; vinyl acetate and allyl stearate, cross-linked with 0.2% divinyl benzene; vinyl acetate and 1-octadecene, cross-linked with 0.2% divinyl benzene; and allyl propionate and allyl stearate cross-linked with 0.2% divinyl benzene.

As examples of liposoluble film-forming polymers, mention can be made of copolymers of vinyl ester and at least one other monomer which may be a vinyl ester, in particular: vinyl neodecanoate; vinyl benzoate; vinyl t-butyl benzoate; and α-olefin; an alkyl vinyl ether; an allyl or a methallyl ester.

As liposoluble film-forming polymers, mention can also be made of liposoluble copolymers, and in particular those that result from copolymerization of vinyl esters having nine to 22 carbon atoms or acrylates or alkyl methacrylates, the alkyl radicals having ten to 20 carbon atoms.

Such liposoluble copolymers may be selected from the copolymers of: vinyl polystearate; vinyl polystearate cross-linked with the help of divinyl benzene, diallyl ether, or diallyl phthalate; stearyl(meth)acrylate copolymers; vinyl polylaurate; lauryl(meth)acrylate; which (meth)acrylates may be cross-linked with the help of ethylene glycol dimethacrylate or glycol tetraethylene.

The above-defined liposoluble copolymers are known and in particular they are described in application FR-A-2232303; they may have a mass average molecular weight lying in the range 2,000 to 500,000, and preferably in the range to 4,000 to 200,000.

As liposoluble film-forming polymers usable in the invention, mention can also be made of polyaklylenes and in particular of C₂-C₂₀ alcene copolymers such as: polybutene; alkylcelluloses with a C₁ to C₈ optionally saturated linear or branched alkyl radical such as ethylcellulose and propylcellulose; copolymers of vinyl pyrolidone (VP) and in particular copolymers of vinyl pyrolidone and C₂ to C₄₀ or better C₃ to C₂₀ alcene. As examples of VP copolymers usable in the invention, mention can be made of the following copolymers: VP and vinyl acetate; VP and ethyl methacrylate; butyl polyvinyl pyrolidone (PVP); VP and ethyl methacrylate and methacrylic acid; VP and eicosene; VP and hexadecene; VP and triacontene; VP and styrene; VP and acrylic acid and lauryl methacrylate.

Mention can also be made of silicone resins that are generally soluble or swellable in silicone oils, constituted by cross-linked polyorganosiloxane polymers.

The nomenclature for silicone resins is known under the term “MDTQ”, the resin being described as a function of the different siloxane monomer units it comprises, with each of the letters “MDTQ” characterizing one type of unit.

As examples of commercially available polymethylsilsesquioxane resins, mention can be made of those sold:
by the supplier Wacker under the reference Resin MK such as Belsil PMS MK; and
by the supplier Shin-Etsu under the reference KR-220L.

As siloxysilicate resins, mention can be made of trimethylsiloxysilicate (TMS) resins such as those sold under the reference SR1000 by the supplier General Electric or under the reference TMS 803 by the supplier Wacker. Mention can also be made of the trimethylsiloxysilicate resins sold in a solvent such as cyclomethicone, sold under the name “KF-7312J” by the supplier Shin-Etsu, or under the names “DC 749”, or “DC 593” by the supplier Dow Corning.

Mention can also be made of copolymers of silicone resins such as those mentioned above with polydimethylsiloxanes such as the pressure-sensitive adhesive copolymers sold by the supplier Dow Corning under the reference BIO-PSA and described in U.S. Pat. No. 5,162,410, or indeed silicone copolymers obtained by reaction between a silicone resin such as those described above, and a diorganosiloxane such as those described in document WO 2004/073626.

In an embodiment of the invention, the film-forming polymer is a film-forming linear sequenced ethylene polymer preferably comprising at least a first sequence and at least a second sequence having different glass transition temperatures (Tg), said first and second sequences being connected together by an intermediate sequence comprising at least one monomer constituting the first sequence of at least one monomer constituting the second sequence.

Advantageously, the first and second sequences of the sequenced polymer are mutually incompatible.

Such polymers are described for example in documents EP 1 411 069 and WO 2004/028488.

The film-forming polymer may be selected from block or statistical polymers and/or copolymers comprising in particular: polyurethanes; polyacrylics; silicones; fluorinated polymers; butyl gums; ethylene copolymers; natural gums; polyvinyl alcohols; and mixtures thereof. The monomers of the block or statistical copolymers including at least one association of monomers for which the polymer has a glass transition temperature lower than ambient temperature (25° C.) can be selected in particular from: butadiene; ethylene; propylene; acrylic; methacrylic; isoprene; isobutene; silicone; and mixtures thereof.

The film-forming polymer may also be present in the composition in the form of particles in dispersion in an aqueous phase or in a non-aqueous solvent phase, generally known as a latex or a pseudolatex. Techniques for preparing such dispersions are well known to the person skilled in the art.

The composition of the invention may include a plasticizing agent encouraging the film-forming polymer to form a film. Such a plasticizing agent may be selected from all of the compounds known to the person skilled in the art as being suitable for performing the looked-for function.

Naturally, this list of polymers is not exhaustive.

Preferably, when the medium containing the monodisperse particles contains a film-forming polymer, the film-forming polymer is, for example, an aqueous dispersion of an acrylic, vinyl, fluorinated, or silicone polymer, or of a mixture thereof.

The percentage by weight of film-forming polymer in the composition containing the monodisperse particles may lie for example in the range 0.1% to 10%.

When the composition containing the monodisperse particles contains a polymer that is not fully polymerized and/or cross-linked, the polymerization and/or cross-linking can be undertaken by thermal triggering or by using ultraviolet radiation.

Polymerization can also be performed by adding an initiator and possibly a cross-linking agent.

When it is desired to make a lattice of monodisperse particles in the medium, it is possible to add a monomer and an initiator and possibly also a cross-linking agent, and then to carry out polymerization.

The polymerization may take place when the formulation is fabricated or else after it has been applied to the skin. This method makes it possible to produce polymers of large molecular mass or cross-linked polymers. This makes it possible to vary at will the rheology of the resulting system.

The medium may also include a polymer enabling a gel to be formed, e.g. before or after the composition is applied on the substrate to be made up.

### Polymers Enabling a Gel to be Formed

Forming a gel can serve, for example, to improve the cohesion of the lattice of monodisperse particles and/or to make it responsive to an external stimulus and/or to the concentration of a compound in the medium, e.g. the concentration in water.

The polymer enabling a gel to be formed may be selected from cellulose derivatives, alginates and their derivatives, in particular their derivatives such as propylene glycol alginate, or their salts such as sodium alginate, calcium alginate, derivatives of polyacrylic acid or polymethacrylate acid, polyacrylamide derivatives, polyvinylpyrrolidone derivatives, derivatives of ether or of polyvinyl alcohol, and mixtures thereof, amongst others.

The polymer may be selected in particular from derivatives of modified cellulose, e.g. selected from: carboxymethylcellulose, soda carboxymethycellulose, carboxymethyl-hydroxyethylcellulose, carboxyethylcellulose, hydroxyethylcellulose, hydroxyethyl-ethylcellulose, hydroxypropylcellulose, hydroxypropylmethylcelluose, methylcellulose, soda methylcellulose, microcrystalline cellulose, soda cellulose sulfate, and mixtures thereof.

The polymer enabling a gel to be formed may also be selected from natural polymer derivatives, such as for example: gelatin and glucomannane and galactomannane polysaccharides extracted from seeds, vegetable fibers, fruits, seaweed, starch, plant resins, or indeed it may be of microbial origin.

The quantity by weight of polymer for forming a gel in the composition may lie in the range 0.5% to 40%, better in the range 1% to 20%.

The polymer for forming a gel may polymerize after the composition has been applied on the substrate to be made up. In a variant, the gel is formed before the composition is applied on keratinous materials, and the composition is then applied thereto.

Hydrogels can be obtained from acrylamide, acrylic, or vinylpyrrolidone monomers, for example. An example of a hydrogel obtained by this method based on N-isopropylacrylamide polymerized under a UV lamp in a colloidal crystal of polystyrene is described for example in patent WO 98/41859. The article by Foulger et al., Advanced Materials, 13, 1898-1901 (2001) describes a hydrogel based on polyethylene glycol methacrylate and dimethacrylate.

The gel may also be formed prior to fabricating the composition. For example it is possible to make an oily gel based on polydimethylsiloxane elastomer from a lattice of polystyrene spheres as described in the article by H. Fudouzi et al., Langmuir, 19, 9653-9660 (2003).

### Fatty Phase

Although the composition containing the monodisperse particles need not have any oil, it is nevertheless possible for the composition of the invention to include a fatty phase in certain embodiments. The monodisperse particles may optionally be contained in this fatty phase.

In particular, the fatty phase may be volatile.

One or more oils may be included in such a manner as to avoid losing the looked-for spectral reflectance or coloration effect.

The composition may include an oil such as for example: synthetic ethers and esters; linear or branched hydrocarbons, of mineral or synthetic origin; fatty alcohols having eight to 26 carbon atoms; partially fluorinated hydrocarbon and/or silicone oils; optionally-volatile silicone oils such as polymethylsiloxanes (PDMS) having a linear or a cyclic silicone chain that are liquid or pasty at ambient temperature; and mixtures thereof, other examples being given below.

A composition in accordance with the invention may include at least one volatile oil.

### Volatile Oils

In the meaning of the present invention, the term “volatile oil” is used to mean an oil (or non-aqueous medium) suitable for evaporating on contact with the skin in less than 1 hour, at ambient temperature and at atmospheric pressure.

The volatile oil is a volatile cosmetic oil that is liquid at ambient temperature, in particular having a vapor pressure that is not zero at ambient temperature and at atmospheric pressure, in particular having vapor pressure lying in the range 0.13 pascals (Pa) to 40,000, (10⁻³ millimeters of mercury (mmHg) to 300 mmHg), in particular lying in the range 1.3 Pa to 13,000 Pa (0.01 mmHg to 100 mmHg), and more particularly lying in the range 1.3 Pa to 1300 Pa (0.01 mmHg to 10 mmHg).

The volatile hydrocarbon oils can be selected from hydrocarbon oils of animal or vegetable origin having eight to 16 carbon atoms, and in particular C₈-C₁₆ branched alkanes (also known as isoparaffins) such as isododecane (also known as 2,2,4,4,6-pentamethyl heptane); isodecane; isohexadecane; and for example the oils sold under the trade names Isopars® or Permethyls®.

As volatile oils, it is also possible to use volatile silicone oils, in particular volatile linear or cyclic silicone oils, in particular those having viscosity≦8 centistokes (cSt) (8×10⁻⁶ square meters per second (m²/s)), and having in particular two to ten silicon atoms, and more specifically two to seven silicon atoms, such silicones optionally including alkyl or alkoxy groups with one to ten carbon atoms. As volatile silicone oils usable in the invention, mention can be made in particular of: dimethicones having viscosity in the range 5 cSt to 6 cSt; octamethyl cyclotetrasiloxane; decamethyl cyclopentasiloxane; dodecamethyl cyclohexasiloxane; heptamethyl hexyltrisiloxane; heptamethyloctyl trisiloxane; hexamethyl disiloxane; octamethyl trisiloxane; decamethyl tetrasiloxane; dodecamethyl pentasiloxane; and mixtures thereof.

It is also possible to use fluorinated volatile oils such as nonafluoromethoxybutane or perfluoromethylcyclo-pentane, and mixtures thereof.

It is also possible to use a mixture of the above-mentioned oils.

### Non-Volatile Oils

The composition containing the monodisperse particles may contain a non-volatile oil.

In the meaning of the present invention, the term “non-volatile oil” is used to mean an oil having a vapor pressure of less than 0.13 Pa, and in particular oils of high molecular mass.

The non-volatile oils may in particular be selected from hydrocarbon oils, fluorinated where appropriate, and/or non-volatile silicone oils.

As non-volatile hydrocarbon oils that can be suitable for implementing the invention, mention can be made in particular of:
hydrocarbon oils of animal origin;
hydrocarbon oils of vegetable origin such as: phytostearyl esters such as phytostearyl oleate, phytostearyl isostearate, and lauroyl, octyldodecyl, phytostearyl glutanate, e.g. sold under the name Eldew PS203 by Ajinomoto; triglycerides constituted by esters of fatty acids and glycerol in which the fatty acid may have chain lengths varying in the range C₄ to C₂₄, which chains may be linear or branched, saturated or unsaturated; these oils are in particular heptanoic or octanoic triglycerides; any of the following oils: wheat germ; sunflower; grape pip; sesame; maize; apricot; castor bean; karite; avocado; olive; soybean; sweet almond; palm; rapeseed; cottonseed; hazel nut; macademia nut; jojoba; alfalfa; poppyseed; squash; vegetable marrow; black current; evening primrose; millet; barley; quinoa; rye; safflower; candlenut; passion flower; and muskat rose; also karite butter; or indeed caprylic/capric acid triglycerides such as those sold by the supplier Stéarineries Dubois or those sold under the names Miglyol 810®, 812®, and 818® by the supplier Dynamit Nobel;
hydrocarbon oils of mineral or synthetic origin such as, for example:
   synthetic ethers having 10 to 40 carbon atoms;
   linear or branched hydrocarbons of mineral or synthetic origin such as: Vaseline; polydecenes; hydrogenated polyisobutene such as: parleam; squalane; and mixtures thereof, and in particular hydrogenated polyisobutene;
   synthetic esters such as oils having the formula R₁COOR₂ in which R₁ represents the residue of a linear or branched fatty acid having one to 40 carbon atoms, and R₂ represents a hydrocarbon chain, in particular a branched chain having one to 40 carbon atoms and satisfying the condition that R₁+R₂ is ≧10.

Esters may be selected in particular from esters of fatty acids in particular, such as for example:
cetostearyle octanoate; esters of isopropyl alcohol, such as: isopropyl myristate; isopropyl palmitate; ethyl palmitate; 2-ethyl-hexyl palmitate; stearate or isopropyl stearate; isostearyl isostearate; octyl stearate; hydroxyi esters such as isostearyl lactate; octyl hydroxystearate; diisopropyl adipate; heptanoates; and in particular isostearyl heptanoates; octanoates; decanoates; or ricinoleates of alcohols or of polyalcohols such as: propylene glycol dioctanoate; cetyl octanoate; tridecyl octanoate; 4-diheptanoate; and ethyl 2-hexyl palmitate; alkyl benzoate; polyethylene glycol diheptanoate; propyleneglycol diethyl 2-hexaonate; and mixtures thereof; C₁₂ to C₁₅ alcohol benzoates; hexyl laurate; neopentanoic acid esters such as: isodecyl neopentanoate; isotridecyl neopentanoate; isostearyl neopentanoate; octyldocecyle neopentanoate; isononanoic acid esters such as: isnonyl isononanoate; isotridecyl isononanoate; octyl isononanoate; hydroxyl esters such as: isostearyl lactate; diisostearyl malate;
polyol esters and pentaerythritol esters such as dipentaerythritol tetrahydroxystearate or tetraisostearate;
diol dimer and diacid dimer esters such as: Lusplan DD-DA5® and Lusplan DD-DA7®, sold by the supplier Nippon Fine Chemical and described in patent application FR 03/02809;
fatty alcohols that are liquid at ambient temperature having a branched and/or unsaturated carbon chain with 12 to 26 carbon atoms such as: 2-octyldodecanol; isostearyl alcohol; oleic alcohol; 2-hexyldecanol; 2-butyloctanol; and 2-undecylpentadecanol;
higher fatty acids such as: oleic acid; linoleic acid; linolenic acid; and mixtures thereof;
dialkyl carbonates, in which the alkyl 2 chains may be identical or different, such as dicapryl carbonate sold under the name Cetiol CC® by Cognis;
non-volatile silicone oils, such as for example: non-volatile polydimethylsiloxanes (PDMS); polydimethylsiloxanes including alkyl or alkoxy groups that are pendant and/or at the ends of the silicone chain, each group having two to 24 carbon atoms, phenyl silicones such as: phenyl trimethicones; phenyl dimethicones; phenyl trimethylsiloxy diphenylsiloxanes; diphenyl dimethicones; diphenyl methyldiphenyl trisiloxanes; and 2-phenylethyl trimethylsiloxysilicates; dimethicones or phenyltrimethicone of viscosity less than or equal to 100 cSt; and mixtures thereof;
and mixtures thereof.

The composition containing the monodisperse particles need not contain any oil, in particular need not contain any non-volatile oil.

### Kits

The invention also provides kits including a composition of the invention.

These kits have at least one composition for forming the base coat and possibly a top coat.

The kit may thus comprise:
a first composition comprising:
   monodisperse particles;
   a medium enabling an ordered lattice of monodisperse particles to be formed on a substrate on which the composition is applied; and
a second composition including a film-forming polymer.

Such a composition makes it possible to form a base coat and possibly a top coat.

### Base Coat

The base coat is compatible with being applied on keratinous materials, e.g. the skin, the lips, the nails, the eyelashes, or hair, depending on the nature of the makeup desired, in particular one of those mentioned above.

The base coat may include a polymer selected in particular from film-forming polymers.

In various aspects of the invention, the base coat may perform one or more of the following functions:
the base coat may smooth the substrate prior to application of the composition including monodisperse particles so as to facilitate the formation of the first layers of the lattice and obtain a lattice having the largest possible single-crystal zone;
the base coat may color the substrate so as to show up or modify the color produced by the lattice. For this purpose, the base coat may include at least one coloring agent enabling the clarity of the substrate to be diminished. For example the base coat may include a pigment or a colorant that is black or of some other color so as to create a colored background enabling an additional color to be added to the color given by the lattice of monodisperse particles. Amongst the colorants or pigments that may be present in the base coat, mention can be made in particular of: black iron oxide; carbon black; and black titanium dioxide; and
the base coat may improve the adhesion of the composition containing the monodisperse particles on the substrate being made up. For this purpose, the base coat may include at least one polymer presenting properties of being adhesive, or pro-adhesive, i.e. suitable for becoming adhesive by interacting with another compound. In particular, the polymer may present adhesive or pro-adhesive properties in the meaning given in the following patents: FR 2 834 884; FR 2 811 546; and FR 2 811 547.

The base coat may also act on the surface tension of keratinous materials, e.g. so as to ensure good wettability by the coat of composition containing the monodisperse particles, and encouraging the monodisperse particles to stack.

The base coat may include a single polymer that performs at least two of the above-mentioned functions, e.g. the functions of smoothing and of increasing adhesion, and possibly also a coloring function.

The base coat may be formulated as a function of the nature of the monodisperse particles.

In non-limiting embodiments of the invention, the monodisperse particles may be of polystyrene and the base coat may comprise a non-aqueous dispersion (NAD) in isododecane or the Daitosol (Daito Kasei) or Ultrasol (Ganz Chemical) polymers. In other examples, with the monodisperse particles being of silica, the base coat may include an Eastman AQ (20%) or PVA (10%) polymer.

The base coat may include a volatile phase.

The polymer is preferably suitable for forming a film after the composition has been applied and has dried. The film may be formed with the help of a coalescence agent. The polymer may be in dispersion or in solution in an aqueous phase or in an anhydrous phase. The polymer is preferably in dispersion in water or in an oil. Still more preferably, the polymer contains at least one function suitable for ionizing in aqueous solution, such as a carboxylic acid. The polymer is preferably not soluble in contact with an aqueous phase after application and drying.

In this method, it is also possible to use in the base coat monomers or prepolymers that are also suitable for polymerizing after application on the skin, under the action of UV rays, or of heat, or of the presence of water, for example. Examples that can be mentioned are cyanoacrylate monomers and silicone polymers of low molecular mass carrying reactive functions.

As examples of polymers in aqueous dispersion, mention can be made of: Ultrasol 2075 from the supplier Ganz Chemical; Daitosol 5000AD from Daito Kasei; Avalure UR 450 from Noveon; Dynamx from National Starch; Syntran 5760 from Interpolymer; Acusol OP 301 from Rohm & Haas; and Neocryl A 1090 from Avecia.

As examples of polymers in oily dispersion, mention can be made of: NAD and the polymers disclosed in patent application EP-A-1 411 069 in the name of L'Oreal, or the dispersion of acrylic-silicone polymer ACRIT 8HV-1023 from the supplier Tasei Chemical Industries.

The volatile phase may be an aqueous phase or an anhydrous phase.

With an aqueous phase it is preferably constituted by water, alcohol, and glycol.

With an anhydrous phase it is preferably constituted by at least one volatile oil, as defined above.

The base coat may optionally be colored. For a colored base coat, said base coat may contain colorants or pigments. The pigments should preferably be dispersed as finely as possible in order to avoid a rough finish on the formed film.

The base coat may contain other solid components (fillers, effect pigments) or other non-volatile liquid components. The quantities of non-volatile liquid components are preferably small.

### Top Coat

The top coat may, in particular, have the function of changing a visible characteristic such as color or glossiness, and/or the function of improving the retention of the lattice of monodisperse particles on the substrate, in particular of improving the ability of the lattice to withstand friction and avoid crumbling.

The top coat may have one or more polymers optionally capable of penetrating into the lattice of particles, where penetration of a polymer changes the refractive index of the medium around the particles and thus change color.

The top coat may present a volatile phase, which can make it possible to limit changes in color over time, with color changes ceasing once the volatile phase has evaporated.

The second composition may include in particular a volatile oil as defined above.

The top coat may include a non-volatile solvent, which can increase the durability of the color change.

This solvent penetrates into and remains in the medium between the particles, thereby likewise modifying the refractive index around the particles.

The second composition for forming the top coat may thus include a non-volatile oil, as defined above.

The top coat may present a high degree of transparency in order to avoid affecting the color and/or the intensity of the color coming from the lattice of monodisperse particles.

The top coat may also be colored, e.g. for the purpose of exerting an influence on the color and/or the glossiness produced by the lattice of monodisperse particles.

The top coat may also slow down the moistening or drying of the layer of composition that contains the ordered lattice, and can reduce variability over time in the results obtained.

Or on the contrary, the top coat may increase sensitivity to the environment, e.g. for the purpose of making color depend on the ambient humidity or temperature.

The top coat preferably includes a film-forming polymer.

The formulation of the top coat may be adapted to the nature of the monodisperse particles.

In the example of monodisperse particles of silica or of polystyrene, the top coat may comprise a non-aqueous dispersion (NAD) in isododecane. When the monodisperse particles are of polystyrene, the top coat may comprise for example an acrylic copolymer or PVA.

For monodisperse particles of polystyrene, the top coat may comprise, for example, a non-aqueous dispersion (NAD), PVA (10%), or the polymers Eastman AQ (20%), Daitosol, or Ultrasol.

The top coat may contain monodisperse particles of mean size different from the mean size of the monodisperse particles covered by the top coat. This can serve to change the color of the underlying composition.

The top coat may then itself optionally be covered by a layer for improving its retention.

The base coat and the top coat may be present simultaneously, in which case the kit may comprise:
a first cosmetic composition comprising:
   monodisperse particles;
   a physiologically-acceptable medium enabling an ordered lattice of monodisperse particles to be formed on a substrate on which the composition is applied;
a second cosmetic composition for applying onto the substrate before applying the first composition so as to improve adhesion thereof on the substrate and so as to smooth keratinous surfaces; and
a third cosmetic composition for applying onto the first composition in order to change its color and possibly improve the retention of the second composition.

### Additives

The cosmetic composition containing the monodisperse particles, the base coat, and the top coat may include at least one additive selected from the additives that are usual in the field of cosmetics, such as: fillers; hydrophilic or lipophilic gelling agents; hydrosoluble or liposoluble agents; preservatives; moisturizers such as polyols and in particular glycerin; sequestering agents; antioxidants; solvents; fragrances; physical and chemical sunscreens, in particular providing protection against UVA and/or UVB radiation; odor absorbers; pH adjusters (acids or bases); and mixtures thereof.

The additive(s) may be selected in particular from those mentioned in the CFTA Cosmetic Ingredient Handbook, 10th Edition Cosmetic and Fragrance Association Inc., Washington D.C. (2004), incorporated herein by reference.

### Forms

The first composition containing the monodisperse particles may be presented in a variety of forms of the kind used in the field of cosmetics for topical application: direct, inverse, or multiple emulsions, gels, creams, solutions, suspensions, lotions.

The first composition may be in the form of: an aqueous solution or an oily solution, in particular a gelled solution; an emulsion of liquid or semi-liquid consistency of the lotion type, obtained by dispersing a fatty phase in an aqueous phase (O/W) or vice versa (W/O); a triple emulsion (W/O/W or O/W/O); or a suspension or emulsion of soft texture. The same could apply for the second composition for forming the top coat.

The first composition of the invention may constitute a care composition, a makeup composition, and/or a sunscreen composition.

### Method of Applying Makeup

The invention also provides a method of making up keratinous materials, the method comprising the following steps:
applying a base coat on a substrate to be made up; and
applying on the base coat, a cosmetic composition comprising monodisperse particles, and a medium enabling an ordered lattice of monodisperse particles to be formed.

Such a method makes it possible to improve the quality with which the composition containing the monodisperse particles is applied, in particular when said monodisperse particles are in an aqueous medium, and also makes it possible to obtain good “crystallization” after application on the skin or the hair, for example.

As mentioned above, the base coat makes it possible to control and make uniform the surface properties of keratinous materials, in particular surface tension. It also serves to smooth the surface and make its roughnesses uniform. An electrostatic repulsion effect may also take place if the base coat is likely to create an electrostatic charge on contact with water.

Apart from very significantly improving the arrangement of the particles, the base coat may optionally also have the effect of securing the layer of monodisperse particles, making it more stable against external attack.

In this method, the base coat preferably contains a polymer and a volatile phase.

In such a method, the first composition containing the monodisperse particles may comprise an aqueous medium.

As mentioned above the base coat may include a polymer having adhesive properties and/or a coloring agent, in particular of black color.

The composition containing the monodisperse particles may be applied after the base coat has dried, e.g. for a duration greater than or equal to 30 (s).

In another of its aspects, the invention also provides a method comprising the following steps:
applying on a substrate to be made up that is covered in a base coat, a composition comprising monodisperse particles, and a medium enabling an ordered lattice of monodisperse particles to be formed.
applying on the deposit of the composition containing monodisperse particles, a top coat serving to improve the retention of the layer of composition containing the monodisperse particles.

The top coat may include a film-forming polymer, as mentioned above.

The top coat may be applied after the layer of composition containing the monodisperse particles has dried, e.g. over a duration that is greater than or equal to 30 s.

The invention also provides a method in which a first lattice of monodisperse particles having a first mean size is formed, and then a second lattice of monodisperse particles having a mean size different from the first mean size is formed on top of the first lattice.

In another of its aspects, the invention also provides a method in which a lattice of monodisperse particles is formed on keratinous materials and a composition is applied to said lattice enabling the refractive index around the particles of the lattice to be modified, in particular the particles in the surface layer of the lattice, which can make it possible to change the color thereof.

The Applicant has found in particular that it is possible to modify at will the coloring obtained by a first cosmetic composition by using a second composition that is colorless and that is applied subsequently.

The crystal lattice formed by the first composition may be made up of a continuous layer or of discontinuous islands. Light is diffracted by said crystal lattice and the wavelength of the light that is diffracted depends on the distance between the particles and on the refractive index.

The composition that forms the top coat may contain at least one liquid medium suitable for penetrating into the first composition so as to modify the distance between the particles and/or the refractive index. The liquid medium may optionally be volatile. When it is entirely volatile, the color change is temporary and color returns progressively to its initial state. When a large fraction of the liquid medium is non-volatile, it is possible to obtain a durable change in the color.

The crystal lattice may optionally be compact and it may optionally be continuous. It may be formed prior to application or it may form during application.

The composition applied to the lattice of monodisperse particles may contain at least one liquid phase suitable for swelling the lattice or for modifying the refractive index of the medium. When only the refractive index is changed, the liquid phase may have a refractive index that is different from that surrounding the monodisperse particles.

The composition applied to the lattice of monodisperse particles may also contain a polymer so as to fix the first composition.

It is equally possible to use, in the base or top coat, monomers or prepolymers that are also suitable for polymerizing after application on the skin, either under the action of UV rays, or of heat, or of the presence of water, for example. Examples that can be mentioned are cyanoacrylate monomers and silicone polymers of low molecular mass carrying reactive functions.

A colored or non-colored base coat may optionally be applied before these two compositions are applied on the keratinous materials.

### Modes of Application

The composition containing the monodisperse particles, and possibly also the compositions that are to form the base coat and the top coat, may be applied by using an applicator, preferably a flocked applicator, e.g. a flocked foam or tip, or a paint brush, in particular having bristles that are fine and flexible.

Application may be performed differently, for example by means of: a foam; a felt; a spatula; a sintered piece; a brush; a comb; or a woven or non-woven fabric.

Application can also be done with a finger or by depositing the composition directly on the substrate to be treated, for example by spraying, e.g. with the help of a piezoelectric device, or by transferring a layer of composition that has previously been deposited on an intermediate substrate.

The composition containing the monodisperse particles may be applied with a thickness for example in the range 1 μm to 10 μm, better in the range 2 μm to 5 μm.

By way of example, the composition containing the monodisperse particles may be applied at a density lying in the range 1 milligram per square centimeter (mg/cm²) to 5 mg/cm².

The lattice of monodisperse particles that form comprises, for example, at least six layers of particles, and better six to 20 layers of particles.

The composition may be applied on keratinous materials in such a manner as to enable the lattice of monodisperse particles to form after deposition. Thus, the medium of the composition may be formulated in such a manner that evaporation of the solvent(s) it contains takes place sufficiently slowly to allow the particles enough time to become ordered and also to limit any risk of particles clumping together in disordered manner prior to application.

By way of example, the top coat is applied over a thickness lying in the range 0.5 μm to 10 μm. The base coat may be applied, for example, over a thickness lying in the range 0.5 μm to 10 μm.

The top coat may be applied by spraying.

### Packaging

The first and second composition may be packaged in any receptacle or on any substrate provided for this purpose.

The kit may comprise a first receptacle containing the composition including the monodisperse particles, and a second receptacle containing at least one of the compositions for forming the base coat and the top coat.

### PROPOSED EXAMPLES

The quantities are by weight.

### Example 1

### Makeup Kit

### a) Base Coat:

| | |
|---|---|
| Ultrasol ® 2075 (Ganz Chemical)* | 90% |
| Water | 10% |

*Copolymer of acrylate and ammonium methacrylate in dispersion in water at a concentration of 50% by weight.

### b) Composition Containing the Monodisperse Particles:

| | |
|---|---|
| Silica monodisperse particles (mean size 220 nm) | 30% |
| Ultrapure water | 70% |

This composition was prepared by diluting the Seahoster® KE-W20 product from Nippon Shokubai (monodisperse silica having a mean size of 220 nm at a concentration of 20% in water, with conductivity 180 μS.cm⁻¹) to a concentration of 10% by weight in ultrapure water (conductivity 0.5 μS.cm⁻¹), and then centrifuging the mixture for 30 min at a speed of 4000 rpm. The supernatent was removed after centrifuging. The final concentration of silica particles was adjusted to be 30% by weight.

The base coat a) was applied initially (at a rate of 2.5 mg.cm⁻²), e.g. on the skin, and then after drying for a few minutes, composition b) was applied (at a rate of 3 mg.cm⁻²).

Application was performed with a flocked endpiece, for example.

The resulting color varied from violet to green depending on the angle of observation, being predominantly blue.

### Example 2

### Makeup Kit

The base coat was identical to that of Example 1.

The composition coating the monodisperse particles was prepared by replacing the Seahoster® KE-W20 product from the supplier Nippon Shokubai with its Seahoster® KE-W25 product (monodisperse silica having a mean size of 250 nm at a concentration of 20% in water, with conductivity of 55 μS.cm⁻¹), using an identical technique.

The base coat was initially applied (at a rate of 2.5 mg.cm⁻²), e.g. on the skin, and then after drying for a few minutes the composition containing the monodisperse particles was applied on the base coat (at a rate of 2.5 mg.cm⁻²).

The application was performed using a flocked endpiece, for example.

The resulting color varied from blue to yellow depending on the angle of observation, and was predominantly green.

### Example 3

### Makeup Kit

The base coat was identical to that of Example 1.

The composition containing the monodisperse particles was prepared using the Seahoster® KE-W30 product from the supplier Nippon Shokubai (monodisperse silica having a mean size of 309 nm in the form of a powder, with CV=4.5%), and having the following formulation:

| | |
|---|---|
| Seahoster ® KE-P30 | 30% |
| Water | 60% |
| Ethylene glycol | 10% |

The base coat was initially applied (at a rate of 2.5 mg.cm⁻²), e.g. on the skin, and then after drying for a few minutes the composition containing the monodisperse particles was applied on the base coat (at a rate of 2.5 mg.cm⁻²).

After application and drying, the resulting color varied depending on the angle of observation, and was predominantly red.

### Example 4

### Makeup Kit

The base coat was identical to that of Example 1.

The composition containing the monodisperse particles had the following formulation:

| | |
|---|---|
| Polystyrene particles (mean size 290 nm) | 30% |
| Acid black 1 ® (CI20470) colorant | 0.2% |
| Water | 69.8% |

The composition was prepared by centrifuging the Optibind® Polystyrene Microparticles product from the supplier Seradyn (monodisperse polystyrene particles having a mean size of 290 nm with CV=3.5% at a concentration of 10% in water, with conductivity of 700 μS.cm⁻¹) for 30 min at a speed of 10,000 rpm. The supernatent was removed after centrifuging. The final polystyrene particle concentration was adjusted to 30% by weight by adding ultrapure water containing a black colorant and by redispersing the composition using an ultrasound vessel.

The base coat was initially applied (at a rate of 2.5 mg.cm⁻²), e.g. on the skin, and then after drying for a few minutes the composition containing the monodisperse particles was applied on the base coat (at a rate of 2 mg.cm⁻²).

After application and drying, the resulting color varied depending on the angle of observation, and was predominantly red.

### Example 5

### Makeup Kit

The base coat was identical to Example 1.

The composition containing the monodisperse particles had the following formulation:

| | |
|---|---|
| Monodisperse polystyrene (mean size 290 nm) | 30% |
| Ultrasol ® 2075C (Ganz Chemical) | 2% |
| Water | 63% |

This composition was prepared by centrifuging the Optibind® Polystyrene Microparticles product from the supplier Seradyn (monodisperse polystyrene having a mean size of 290 nm with CV=3.5% at a concentration of 10% in water, with conductivity of 700 μS.cm⁻¹) for 30 min at a speed of 10,000 rpm. The supernatent was removed after centrifuging. The final polystyrene particle concentration was adjusted to 30% by weight by adding ultrapure water containing the polymeric dispersion of Ultrasol® 2075C from the supplier Ganz Chemical.

The base coat was initially applied (at a rate of 2.5 mg.cm⁻²), e.g. on the skin, and then after drying for a few minutes the composition containing the monodisperse particles was applied on the base coat (at a rate of 2 mg.cm⁻²).

After application and drying, the resulting color varied depending on the angle of observation, and was predominantly red. The presence of the polymer in the composition containing the monodisperse particles improved the ability of the deposit to withstand rubbing.

### Example 6

### Makeup Kit

### a) Base Coat:

| | |
|---|---|
| Neocryl ® 1097 (Avecia)¹ | 75% |
| Water | 8.5% |
| Ethanol | 5% |
| Nuvis ® FX1100 (Elementis) | 0.5% |
| Black iron oxide | 1% |
| Butylene glycol | 5% |
| N-butyl ether propylene glycol | 2.5% |
| N-butyl ether dipropylene glycol | 2.5% |

¹Styrene and acrylic copolymer in dispersion in water at a concentration of 48%.
²Steareth-100/PEG-136/HMDI Copolymer.

The base coat was obtained as follows:

The iron oxide was initially ground in the butylene glycol.

The water and then the other components were added with stirring to the polymer aqueous dispersion.

### b) Composition Containing the Monodisperse Particles

The composition containing the monodisperse particles was identical to that of Example 2.

The base coat was initially applied (at a rate of 2.5 mg.cm⁻²), e.g. on the skin, and then after drying for a few minutes the composition containing the monodisperse particles was applied on the base coat (at a rate of 3 mg.cm⁻²).

The color obtained was a very bright green.

### Example 7

### Makeup Kit

The aqueous base coat and the composition containing the monodisperse particles were identical to those of Example 2.

### Top Coat

The top coat was prepared as follows:

A dispersion of copolymers of methyl acrylate and acrylic acid was prepared at a ratio of 95/5 in isododecade using the method of Example 1 of document EP-A-0 749 746 incorporated herein by reference, replacing heptane by isododecane. A dispersion was obtained in a isododecane of particles that were surface stabilized by a sequenced two-block copolymer of polystyrene and copoly(ethylene-propylene) sold under the name Kraton® G1701 (Shell), having a dry matter content of 24.6% by weight. The copolymer is a film-forming copolymer.

The base coat was initially applied (at a rate of 2.5 mg.cm⁻²), e.g. on the skin, and then after drying for a few minutes the composition containing the monodisperse particles was applied on the base coat (at a rate of 2.5 mg.cm⁻²). After drying completely, the top coat was applied (at a rate of 1 mg.cm⁻²)

Very bright green coloring was obtained that presented very good durability.

### Example 9

### Makeup Kit

### a) Base Coat:

| | |
|---|---|
| Ultrasol ® 2075 (Ganz Chemical)* | 80% |
| Cab-O-Jet 200 Black Colorant** | 10% |
| Water | 10% |

*Copolymer of acrylate and ammonium methacrylate in dispersion in water at a concentration of 50% by weight.
**Carbon black having a size of 130 nm in aqueous dispersion at 20% sold by the supplier Cabot Corp.

### b) Composition Containing the Monodisperse Particles:

| | |
|---|---|
| Seahoster ® KE-P30 (Nippon Shokubai)* | 30% |
| Water | 60% |
| Ethylene glycol | 10% |

*Monodisperse silica having a mean size of 309 nm in the form of a powder, with CV = 4.5%.

The base coat was initially applied (at a rate of 2.5 mg.cm⁻²), e.g. on the skin, and then after drying for a few minutes the composition containing the monodisperse particles was applied on the base coat (at a rate of 2.5 mg.cm⁻²).

After application and drying, the resulting color varied depending on the angle of observation, and was predominantly red. Application was performed with a flocked endpiece, for example.

The color obtained is much more vivid and not as off-white as without a base coat. The presence of the base coat also improved the ability of the second coat to withstand rubbing.

### Example 10

### Makeup Kit

### a) Base Coat:

The base coat was prepared as follows:

A dispersion of copolymers of methyl acrylate and acrylic acid was prepared at a ratio of 95/5 in isododecade using the method of Example 1 of document EP-A-0 749 749, replacing heptane by isododecane. A dispersion was obtained in a isododecane of particles that were surface stabilized by a sequenced two-block copolymer of polystyrene and copoly(ethylene-propylene) sold under the name Kraton® G1701 (Shell), having a dry matter content of 24.6% by weight. The copolymer is a film-forming copolymer.

### b) Composition Containing the Monodisperse Particles:

| | |
|---|---|
| Polystyrene (PS) monodisperse particles of size | 30% |
| 290 nm* | |
| Water | 70% |

*Optibind PS microparticles sold by the supplier Seradyn (CV = 3.5%)

The base coat was initially applied (at a rate of 2.5 mg.cm⁻²), e.g. on the skin, and then after drying for a few minutes the composition containing the monodisperse particles was applied on the base coat (at a rate of 2.5 mg.cm⁻²). After drying completely, the initial composition can be applied as a top coat (at a rate of 1 mg.cm⁻²)

Very bright red coloring was obtained that presented very good durability.

### Example 11

### Makeup Composition

The composition containing the monodisperse particles had the following formulation:

| | |
|---|---|
| Monodisperse polystyrene (mean size 290 nm)* | 30 |
| Ultrasol ® 2075C (Ganz Chemical) | 2.5% |
| Nuvis ® FX1100 (Elementis) | 0.5% |
| Ethanol | 1.5% |
| Ethylene glycol (EG) | 3.5% |
| Water | 62% |

*Optibind Polystyrene Microparticles by Seradyn (CV =3.5%)

The base coat constituted by Ultrasol® 2075C (Ganz Chemical) was initially applied (at a rate of 2.5 mg.cm⁻²), e.g. on the skin, and then after drying for a few minutes the composition containing the monodisperse particles was applied on the base coat (at a rate of 2.5 mg.cm⁻²).

After application and drying, the resulting color varied depending on the angle of observation, and was predominantly red. The presence of the polymer in the composition containing the monodisperse particles improved the ability of the deposit to withstand rubbing.

### Examples 12 and 13

### Makeup Kits with Top Coat

### a) Base Coat:

| | |
|---|---|
| Ultrasol ® 2075 (Ganz Chemical)* | 80% |
| Cab-O-Jet 200 Black Colorant** | 10% |
| Water | 10% |

*Copolymer of acrylate and ammonium methacrylate in dispersion in water at a concentration of 50% by weight.
**Carbon black having a size of 130 nm in aqueous dispersion at 20% sold by the supplier Cabot Corp.

### b) Composition Containing the Monodisperse Particles:

| | |
|---|---|
| Monodisperse polystyrene particles (216 nm)* | 30% |
| Water | 70% |

*Optibind Polystyrene Particles sold by the supplier Seradyn (the polystyrene particles have been concentrated after centrifuging in order to reach the desired concentration).

### c) Compositions Modifying the Color

### Composition A (Example 12)

| | |
|---|---|
| Eastman AQ 55 S1 | 20% |
| Water | 80% |

¹Polyester sold by the supplier Eastman Chemical.

### Composition B (Example 13)

| | |
|---|---|
| Sylgard 184 Silicone Elastomer Base | 50% |
| Sylgard 184 Silicone Elastomer Curing Agent | 5% |
| Hexamethyldisiloxane | 45% |

The initially-blue composition becomes blue-green after composition A or B has been applied and has dried.

### Example 14

### Photoprotective Kit with a Base Coat

### a) Base Coat:

| | |
|---|---|
| Ultrasol ® 2075 (Ganz Chemical)* | 80% |
| Tioveil AQ G** | 10% |
| Water | 10% |

*Copolymer of acrylate and ammonium methacrylate in dispersion in water at a concentration of 50% by weight.
**Dispersion of titanium nano-oxide in water sold by the supplier Uniqema.

### b) Photoprotective Composition Containing the Monodisperse Particles:

| | |
|---|---|
| Monodisperse silica particles (mean size 100 nm)* | 30% |
| Mexoryl SX** | 10% |
| Ultrasol ® 2075 (Ganz Chemical) | 3% |
| Water | 57% |

*Seahostar W10 by Nippon Shokubai (the silica particles have been concentrated after centrifuging in order to reach the desired concentration).
**UVA filter in aqueous solution sold by the supplier Chimex.

The composition containing the monodisperse particles is applied after the composition forming the base coat has been applied and has dried. This composition kit provides good protection over the entire UV range.

Naturally, the invention is not limited to the examples described. In particular, other coloring agents can be added.

The term “comprising a” should be understood as being synonymous with “comprising at least one” unless specified to the contrary.

The term “lying in the range” should be understood as including the limits of the range, unless specified to the contrary.

1-34. (canceled)

35. A kit comprising:
a first cosmetic composition comprising:
a physiologically-acceptable medium; and
monodisperse particles suitable for forming an ordered lattice of monodisperse particles on a substrate on which the composition is applied; and
a second cosmetic composition including a polymer, said second composition being for application under a layer of first composition.

36. A kit according to claim 35, the second composition including a film-forming polymer.

37. A kit according to claim 35, the second composition containing at least one coloring agent.

38. A kit according to claim 37, the coloring agent being carbon-black.

39. A kit according to claim 38, the second composition being black.

40. A kit according to claim 35, the first composition comprising an aqueous medium, the particles being contained in an aqueous phase.

41. A kit according to claim 35, the first composition comprising an aqueous medium.

42. A kit according to claim 35, the polymer being in dispersion in an aqueous phase.

43. A kit according to claim 35, the first composition including a polymer containing at least one function suitable for ionizing in aqueous solution.

44. A kit according to claim 35, the second composition including a polymer that is not soluble in contact with an aqueous phase after application and drying.

45. A kit according to claim 35, the second composition including a polymer in dispersion in an anhydrous phase.

46. A kit according to claim 45, the anhydrous phase including a volatile oil.

47. A kit according to claim 46, the volatile oil being selected from a volatile silicone and isododecane.

48. A kit according to claim 35, the monodisperse particles having a mean size lying in the range 80 nm to 500 nm.

49. A kit according to claim 35, in which the mean size of the monodisperse particles lies in the range 190 nm to 310 nm.

50. A kit according to claim 35, the percentage by weight of monodisperse particles in the first composition being greater than or equal to 15%.

51. A kit according to claim 50, the percentage by weight being greater than or equal to 20%.

52. A kit according to claim 35, the coefficient of variation (CV) in the size of the monodisperse particles being less than or equal to 5%.

53. A kit according to claim 35, the monodisperse particles including an inorganic compound.

54. A kit according to claim 53, the inorganic compound being silica.

55. A kit according to claim 35, the monodisperse particles including an organic compound.

56. A kit according to claim 55, the monodisperse particles including a polymer selected from: polystyrene (PS); polymethyl methacrylate (PMMA); polyacrylamide; and mixtures and derivatives thereof.

57. A kit according to claim 35, the first composition including an additional coloring agent.

58. A kit according to claim 57, the additional coloring agent including an effect pigment.

59. A kit according to claim 35, the medium of the first composition presenting a relative dielectric constant that is greater than or equal to 10.

60. A kit according to claim 35, the medium of the first composition including at least one compound presenting a —OH bond.

61. A kit according to claim 35, the medium of the first composition including at least one polymer enabling a gel to be formed.

62. A kit according to claim 35, the medium of the first composition including at least one film-forming polymer.

63. A kit according to claim 35, including a third cosmetic composition for application on the first composition.

64. A kit comprising:
a first composition comprising:
monodisperse particles; and
a physiologically-acceptable medium enabling an ordered lattice of monodisperse particles to be formed on a substrate on which the composition is applied; and
a second composition including at least one coloring agent.

65. A kit according to claim 64, the coloring agent comprising a pigment or a colorant that is black.

66. A method of applying makeup, the method comprising the steps consisting in:
applying a base coat containing a polymer, in particular a film-forming polymer, on a substrate to be made up; and
applying on the base coat, cosmetic composition comprising a physiologically-acceptable medium and monodisperse particles that are suitable for forming an ordered lattice of monodisperse particles.

67. A method according to claim 66, the base coat including a polymer having adhesive or pro-adhesive properties.

68. A method according to claim 66, the base coat including a coloring agent.

69. A method according to claim 68, the coloring agent being black.

70. A method according to claim 66, the composition containing the monodisperse particles being applied after the base coat has dried.

71. A method according to claim 67, the base coat including a coloring agent.

72. A method according to claim 71, the coloring agent being black.
